(19) United States
(12) Patent Application Publication
(10) Pub. No.: US 2004/0064902 A1
(43) Pub. Date: Apr. 8, 2004

(54) Oxidatiton dyeing composition based on 1-(4-aminophenyl) pyrrolidines substituted in position 2 and 5

(76) Inventors: Stephane Sabelle, Paris (FR); Eric Terranova, Magagnosc (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/433,408

(22) PCT Filed: Nov. 13, 2001
(86) PCT No.: PCT/FR01/03540

(30) Foreign Application Priority Data
Dec. 6, 2000 (FR) 00/15840

(51) Int. Cl. A61K 7/13

(52) U.S. Cl. 8/405

(57) ABSTRACT

The subject of the invention is a composition for the oxidation dyeing of keratinous fibers, and in particular human keratinous fibers such as hair, comprising, as oxidation base, a 1-(4-aminophenyl)pyrrolidine substituted at the 2- and 5-positions.

The subject of the invention is also the method for the oxidation dyeing of keratinous fibers using these compositions.

The subject of the invention is a composition for the oxidation dyeing of keratinous fibers, and in particular human keratinous fibers such as hair, comprising, as oxidation base, a 1-(4-aminophenyl)pyrrolidine substituted at least at the 2- and 5-positions.

It is known to dye keratinous fibers, and in particular human hair, with dyeing compositions containing oxidation dye precursors, in particular para-phenylenediamines, ortho- or para-aminophenols, heterocyclic compounds such as diaminopyrazole derivatives, pyrazolo[1,5-a]pyrimidine derivatives, pyrimidine derivatives, pyridine derivatives, 5,6-dihydroxyindole derivatives, 5,6-dihydroxyindoline derivatives generally called oxidation bases. Oxidation dye precursors, or oxidation bases, are colorless or weakly colored compounds which, combined with oxidizing products, can give rise, by a process of oxidative condensation, to colored and coloring compounds.

It is also known that the shades obtained with these oxidation bases can be varied by combining them with couplers or color modifiers, the latter being chosen in particular from aromatic meta-diamines, meta-aminophenols, meta-hydroxyphenols and certain heterocyclic compounds such as, for example, pyrazolo[1,5-b]-1,2,4-triazole derivatives, pyrazolo[3,2-c]-1,2,4-triazole derivatives, pyrazolo[1,5-a]pyrimidine derivatives, pyridine derivatives, pyrazol-5-one derivatives, indoline derivatives and indole derivatives.

The variety of the molecules used in oxidation bases and couplers makes it possible to obtain a rich palette of colors.

The so-called “permanent” color obtained using these oxidation dyes must moreover meet a number of requirements. Thus, it must be without drawbacks from the toxicological point of view, it must make it possible to obtain shades in the desired intensity and exhibit good resistance to external agents (light, adverse weather conditions, washing, permanent waving, perspiration, rubbing).

The dyes must also make it possible to cover gray hair, and be the least selective possible, that is to say make it possible to obtain the smallest possible differences in color right along the same keratinous fiber, which may indeed be differently sensitized (i.e. damaged) between its tip and its root. They must also exhibit good chemical stability in the formulations. They must also exhibit a good toxicological profile.

In the field of hair dyeing, para-phenylenediamine and para-toluenediamine are widely used oxidation bases. They make it possible to obtain a variety of shades with oxidation couplers.

However, a need exists to discover novel oxidation bases exhibiting a better toxicological profile than para-phenylenediamine and para-toluenediamine, while making it possible to confer on the hair excellent properties of color intensity, shade variety, color uniformity and fastness to external agents.

It has already been proposed, in particular in patent application GB 2 239 265, to use 2-(β-hydroxyethyl)-para-phenylenediamine or N,N-bis(β-hydroxyethyl)-para-phenylenediamine as potential replacements for para-phenylenediamine and para-tolylenediamine. It is likewise the case for 2-(hydroxyalkoxy)-para-phenylenediamines (see in particular patent U.S. Pat. No. 5,538,516).

However, N,N-bis(P-hydroxyethyl)-para-phenylenediamine and 2-(β-hydroxyethyl)-para-phenylenediamine have the disadvantage of giving a more limited variety of shades and of giving less color intensity and less uniformity to the hair than para-phenylenediamine and 4-amino-2-methylaniline. It is likewise the case for 2-(hydroxyalkoxy)-para-phenylenediamines which give the hair color which evolves and changes over time.

Moreover, it is known from the literature (R. L. Bent et al., J.A.C.S. 73, 3100, 1951) that 4-para-phenylenediamine derivatives in which one of the nitrogen atoms is contained in a nonaromatic 6- or 7-membered carbon or heterocyclic ring are less oxidizable than 4-para-phenylenediamine derivatives in which one of the nitrogen atoms is substituted with two disymmetric substituents, which are themselves less oxidizable than para-phenylenediamine derivatives in which one of the nitrogen atoms is substituted with two symmetric substituents.

It is also mentioned in the same article that the para-phenylenediamine derivative in which one of the nitrogen atoms is contained in a nonaromatic 5-membered carbon ring is more oxidizable than each of the derivatives cited above. This particular class of N-(4-aminophenyl)-pyrrolidine derivatives therefore makes it possible to obtain condensation reactions with couplers in an oxidizing medium whose kinetics is accelerated compared with the para-phenylenediamine derivatives cited above.

However, oxidation bases which are too oxidizable and which react with couplers according to accelerated reaction rates generally lead to the formation of dyes outside the keratinous fiber. The intensities, the fastness and the uniformity of the colorations thus obtained on the hair are generally insufficient.

However, patent U.S. Pat. No. 5,851,237 proposes the use of 1-(4-aminophenyl)pyrrolidine derivatives optionally substituted on the benzene nucleus in order to replace para-phenylenediamine. In this regard, the same patent proposes very preferentially the use of 1-(4-aminophenyl)pyrrolidone as the substitute for para-phenylenediamine.

Now, it is known from the literature that 1-(4-aminophenyl)pyrrolidine possesses a high allergenic activity (R. L. Bent et al., J.A.C.S. 73, 3100, 1951).

Patent U.S. Pat. No. 5,993,491 proposes the use of N-(4-aminophenyl)-2-hydroxymethylpyrrolidine derivatives optionally substituted on the benzene nucleus and on the pyrrolidine heterocycle at the 4-position with a hydroxyl radical in order to replace para-phenylenediamine. As preferred compounds, said patent proposes N-(4-aminophenyl)-2-(hydroxymethyl)pyrrolidine substituted with a hydrogen atom or a methyl radical at the 3-position. However, it has been clearly established that these compounds do not make it possible to give the hair a coloration of equivalent quality to that obtained with para-phenylenediamine or with para-toluenediamine because of the lack of intensity and of uniformity of the color.

Patent application JP 11158048 proposes hair dyeing compositions which offer good properties of spreading, ease of application and resistance to shampoo. These compositions contain at least one compound chosen from 4-aminoaniline derivatives optionally substituted on the benzene nucleus and in which one of the nitrogen atoms is contained in a 5- to 7-membered carbon ring or at least one compound chosen from 4-aminoaniline derivatives optionally substituted on the benzene nucleus and in which one of the nitrogen atoms is substituted with a radical Z₁ and a radical Z₂, Z₁ being an alkyl, aryl or heterocyclyl group, and Z₂ being a radical —(CH₂—CH₂—O)—Z₃ where Z₃ represents a hydrogen atom, an alkyl, aryl or heterocyclyl group.

In terms of dyeing power, ease of application, uniformity of color and resistance to shampoo, this patent application demonstrates that the preferred derivatives N-(3-isopropoxy-4-aminophenyl)-2,5-dimethylpyrrolidine, 1-(3-methyl-4-aminophenyl)-2,5-dihydroxyethylpyrrolidine, N-(3-methyl-4-aminophenyl)-3-(2-hydroxyethyloxy)pyrrolidine and N-(3-methyl-4-aminophenyl)-2-methyl-4-hydroxypyrrolidine behave like oxidation bases equivalent to para-phenylenediamine derivatives in which the nitrogen atom is contained in a functionalized 6-membered piperidine ring.

Now, it is known that when one of the nitrogen atoms of the para-phenylenediamine derivatives is contained in a 6-membered ring, in particular a piperidine ring, the activation energy to lead to the corresponding quinoneimine oxidized form is among the highest of the N,N-disubstituted para-phenylene-diamines. Consequently, the oxidative condensation reactions of such bases with or without couplers are kinetically and energetically unfavorable and the dyeing compositions containing such oxidation bases give the hair insufficient properties in terms of intensity and uniformity of color compared with those containing para-phenylenediamine or para-toluene-diamine.

The result is that the compositions containing para-phenylenediamine derivatives having a nitrogen atom contained in a functionalized pyrrolidine ring as described in patent application JP 11158048 do not make it possible to give the hair dyeing results equivalent to those obtained with para-phenylenediamine or para-toluenediamine.

It is therefore clear that there is a real need to discover novel oxidation bases having both a good toxicological profile and properties such that the compositions containing them make it possible to confer on the hair excellent properties of intensity of color, variety of shades, uniformity of color and fastness toward various external attacks to which the hair may be subjected.

The aim of the present invention is to develop novel dyeing compositions which do not have the disadvantages of the oxidation bases of the prior art.

This aim is achieved with the present invention whose subject is a composition for the oxidation dyeing of keratinous fibers, and in particular human keratinous fibers such as hair, comprising, in a medium appropriate for dyeing,

at least one oxidation base chosen from compounds of the following formula (I), and or their addition salts with an acid

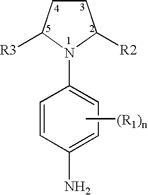

in which:

R₁ represents a halogen atom; a linear or branched C₁-C₇ carbon chain, which is saturated or which may contain one or more double bonds and/or one or more triple bonds, which may be in the form of a 3- to 6-membered ring, it being possible for one or more carbon atoms of the chain to be replaced by an oxygen, nitrogen or sulfur atom, by an SO₂ group or by a halogen atom, the radical R₁ not containing a peroxide bond, or a diazo, nitro or nitroso radical;

R₂ and R₃ represent, independently of each other, a saturated or unsaturated C₁-C₄ carbon chain; a C₁-C₄ alkyl radical substituted with a C₁-C₄ alkoxy radical, an acetoxy radical, an amino radical, a carboxyl radical, a carbamoyl radical, a (C₁-C₄) mono- or dialkylcarbamoyl radical, a (C₁-C₄)alkoxycarbonyl radical, a C₁-C₆ monohydroxyalkoxy radical or with a C₂-C₆ polyhydroxyalkoxy group; a C₁-C₄ monohydroxyalkyl radical; a C₁-C₆ polyhydroxyalkyl radical; a C₁-C₄ aminoalkyl radical in which the amine is mono- or disubstituted with a C₁-C₄ alkyl radical, an acetyl radical, a C₁-C₄ monohydroxyalkyl radical or a C₂-C₆ polyhydroxyalkyl radical; a C₂-C₄ polyaminoalkyl radical; a C₂-C₄ alkyl radical substituted with at least one amino group and at least one hydroxyl group; a carboxyl radical; a carbamoyl radical; a (C₁-C₄) mono- or dialkylcarbamoyl radical; a (C₁-C₄)alkoxycarbonyl radical; a (C₁-C₄)alkylcarbonyl radical

n is between 0 and 2, it being understood that when n is equal to 2, then the radicals R₁ may be identical or different, with the exception of the compound 1-(3-isopropyloxy-4-aminophenyl)-2,5-dimethylpyrrolidine and the compound 1-(3-methyl-4-aminophenyl)-2,5-dihydroxymethyl-pyrrolidine.

Completely unexpectedly and surprisingly, the 1-(4-aminophenyl)pyrrolidine derivatives of formula (I) substituted at least at the 2- and 5-position of the pyrrolidine ring may be used as oxidation dye precursors, and in addition make it possible to obtain dyeing compositions which give intense colorations of keratinous fibers and which exhibit good resistance with respect to external agents (light, adverse weather conditions, washing, permanent waving, perspiration, rubbing). Finally, these compounds are found to be easily synthesizable and are chemically stable.

Unless otherwise stated, the radicals, groups, or carbon chains defined above in formula (I) may be linear or branched.

According to the invention, when it is indicated that one or more of the carbon atoms of the radical R₁ may be replaced by an oxygen, nitrogen or sulfur atom or by an SO₂ group, and/or that said radical R₁ may contain one or more double bonds and/or one or more triple bonds, that means that it is possible, by way of example, to carry out the following conversions:

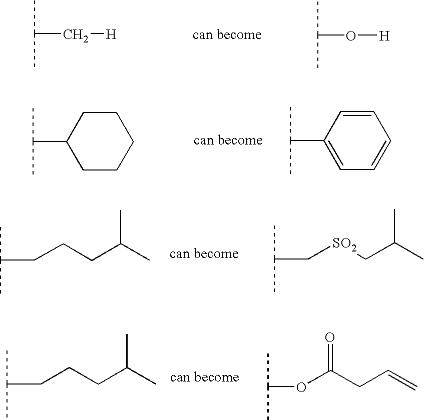

In formula (I), the radical R₁ is preferably chosen from a chlorine or bromine atom, a methyl, ethyl, isopropyl, vinyl, allyl, methoxymethyl, hydroxymethyl, 1-carboxymethyl, 1-aminomethyl, 2-carboxyethyl, 2-hydroxyethyl, 3-hydroxypropyl, 1,2-dihydroxyethyl, 1-hydroxy-2-aminoethyl, 1-amino-2-hydroxyethyl, 1,2-diaminoethyl, methoxy, ethoxy, allyloxy, or 2-hydroxyethyloxy radical. According to a particular embodiment, R₁ is chosen from a methyl, hydroxymethyl, 2-hydroxyethyl, 1,2-dihydroxyethyl, methoxy, or 2-hydroxyethoxy radical, and preferably a methyl, hydroxymethyl or 1,2-dihydroxyethyl radical.

According to a particular embodiment, n is equal to 0 or 1. When n is equal to 1, then R is preferably at the 3-position of the benzene ring.

The radicals R₂ and R₃ of formula (I) are preferably chosen from the hydroxymethyl radical, aminomethyl radical, carboxyl radical, carbamoyl radical, 2-hydroxyethyloxymethyl radical, 2-hydroxyethylaminomethyl radical, and methoxymethyl.

In formula (I), the asymmetric carbons substituted with the radicals R₂ and R₃ may be, independently of each other, of the (R) and/or (S) configuration.

Among the compounds of formula (I) which are used for the present invention, there may be mentioned in particular:

| Formula | Nomeclature | Formula | Nomeclature |
|---|---|---|---|
| 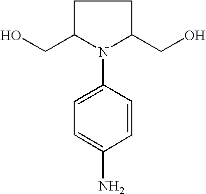 | [1-(4-Aminophenyl)-5- hydroxymethylpyrrolidin-2- yl]methanol | 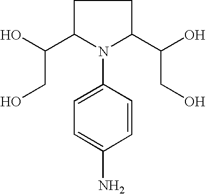 | 1-[1-(4-Aminophenyl)-5- (1,2-dihydroxy- ethyl)pyrrolidin-2-yl]- ethane-1,2-diol |
| 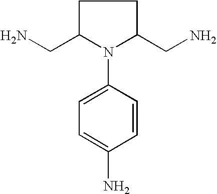 | 4-(2,5-Bisamino- methylpyrrolidin-1-yl)- phenylamine | 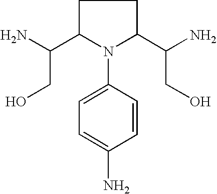 | 2-Amino-2-[5-(1-amino-2- hydroxyethyl)-1-(4- aminophenyl)pyrrolidin-2- yl]ethanol |
| 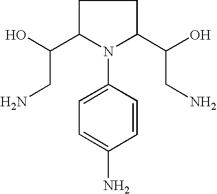 | 2-Amino-1-[5-(2-amino-1- hydroxyethyl)-1-(4- aminophenyl)pyrrolidin-2- yl]ethanol | 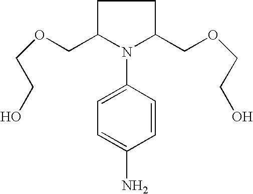 | 2-[1-(4-Aminophenyl)-5-(2- hydroxyethoxy- methyl)pyrrolidin-2- ylmethoxy]ethanol |
| 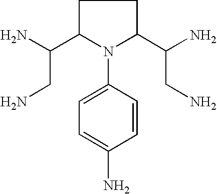 | 1-[1-(4-Aminophenyl)-5- (1,2-diaminoethyl)- pyrrolidin-2-yl]ethane-1,2- diamine | 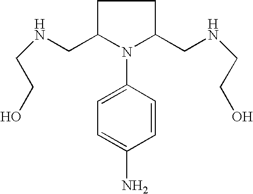 | 2-({1-(4-Aminophenyl)-5- [(2-hydroxyethyl- amino)methyl]pyrrolidin-2- ylmethyl}amino)ethanol |
| 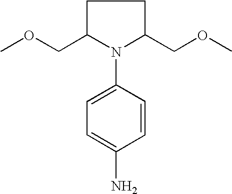 | 4-(2,5-Bismethoxy- methylpyrrolidin-1- yl)phenylamine | 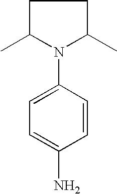 | 4-(2,5-Dimethylpyrroldin- 1-yl)phenylamine |
| 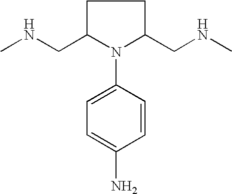 | 4-(2,5-Bismethyl- aminomethylpyrroldin-1- yl)phenylamine | 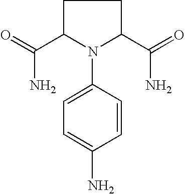 | 1-(4-Aminophenyl)- pyrroldine-2,5-dicarboxylic acid diamide |
| 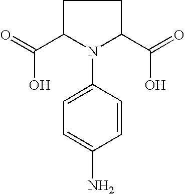 | 1-(4-Aminophenyl)- pyrrolidine-2,5-dicarboxylic acid | 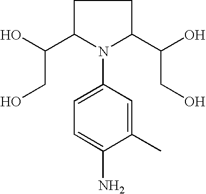 | 1-[1-(4-Amino-3-methyl- phenyl)-5-(1,2- dihydroxyethyl)pyrrolidin- 2-yl]ethane-1,2-diol |
| 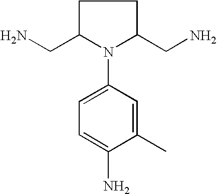 | 4-(2,5-Bisamino- methylpyrrolidin-1-yl)-2- methylphenylamine | 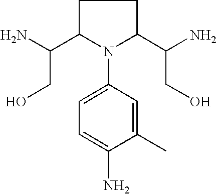 | 2-Amino-2-[5-(1-amino-2- hydroxyethyl)-1-(4-amino- 3-methylphenyl)pyrrolidin- 2-yl)ethanol |
| 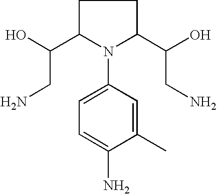 | 2-Amino-1-[5-(2-amino-1- hydroxyethyl)-1-(4-amino- 3-methylphenyl)pyrrolidin- 2-yl)ethanol | 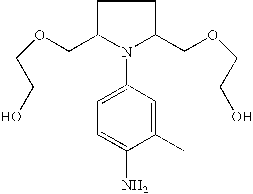 | 2-[1-(4-Amino-3-methyl- phenyl)-5-(2-hydroxy- ethocymethyl)pyrrolidin-2- ylmethoxy)ethanol |
| 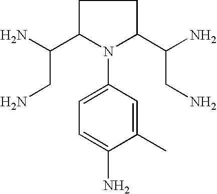 | 1-[1-(4-Amino-3- methylphenyl)-5-(1,2- diaminoethyl)pyrrolidin-2- yl]ethane-1,2-diamine | 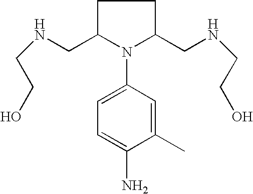 | 2-({1-(4-Amino-3-methyl- phenyl)-5-[(2-hydroxyethyl- amino)methyl]pyrrolidin-2- ylmethyl)amino)ethanol |
| 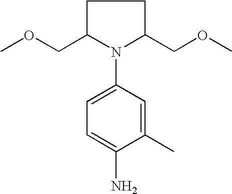 | 4-(2,5-Bismethoxymethyl- pyrrolidin-1-yl)-2-methyl- phenylamine | 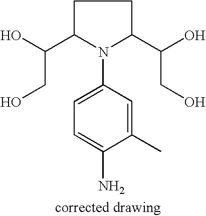 | 1-[1-(4-Amino-3-methyl- phenyl)-5-(1,2-dihydroxy- ethyl)pyrrolidin-2- yl]ethane-1,2-diol |
| 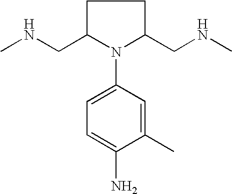 | 4-(2,5-Bismethylamino- methylpyrrolidin-12-yl)-2- methylphenylamine | 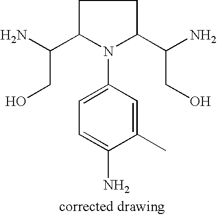 | 2-Amino-2-[5-(1-amino-2- hydroxyethyl)-1-(4-amino- 3-methylphenyl)pyrrolidin- 2-yl]ethanol |
| 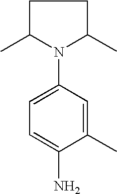 | 4-(2,5-Dimethylpyrrolidin- 1-yl)-2-methylphenylamine | 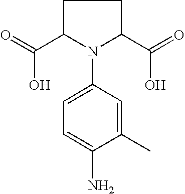 | 1-(4-Amino-3- methylphenyl)pyrrolidin- 2,5-dicarboxylic acid |
| 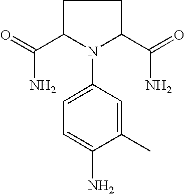 | 1-(4-Amino-3-methyl- phenyl)pyrrolidine-2,5- dicarboxylic acid diamide | | |

and their addition salts with an acid.

The addition salts with an acid of the compounds of formula (I) in accordance with the invention are preferably chosen from the inorganic or organic salts such as the hydrochlorides, hydrobromides, sulfates, citrates, succinates, tartrates, lactates and acetates. The hydrochlorides are particularly preferred.

The para-phenylenediamine derivatives with a pyrrolidinyl group of formula (I), and their methods of synthesis are known; see in particular patent application U.S. Pat. No. DE 4,241,532 (AGFA).

The compound(s) of formula (I) in accordance with the invention preferably represent from 0.0005 to 12% by weight approximately of the total weight of the dyeing composition, and still more preferably from 0.005 to 6% by weight approximately of this weight.

The medium appropriate for dyeing (or carrier) generally consists of water or of a mixture of water and of at least one organic solvent to solubilize the compounds which might not be sufficiently soluble in water. As organic solvent, there may be mentioned for example lower C₁-C₄ alkanols, such as ethanol and isopropanol; polyols or polyol ethers such as 2-butoxyethanol, propylene glycol, monomethyl ether of propylene glycol, monoethyl ether and monomethyl ether of diethylene glycol, as well as aromatic alcohols such as benzyl alcohol or phenoxyethanol, similar products and mixtures thereof.

The solvents may be present in proportions preferably of between 1 and 40% by weight approximately relative to the total weight of the dyeing composition, and still more preferably between 5 and 30% by weight approximately.

The pH of the dyeing composition in accordance with the invention is generally between 3 and 12 approximately, and preferably between 5 and 11 approximately. It can be adjusted to the desired value by means of acidifying or alkalinizing agents normally used in dyeing keratinous fibers.

Among the acidifying agents, there may be mentioned, by way of example, inorganic or organic acids such as hydrochloric acid, orthophosphoric acid, sulfuric acid, carboxylic acids such as acetic acid, tartaric acid, citric acid, lactic acid and sulfonic acids.

Among the alkalinizing agents, there may be mentioned, by way of example, aqueous ammonia, alkali metal carbonates, alkanolamines such as mono-, di- and triethanolamines as well as derivatives thereof, sodium or potassium hydroxides and the compounds of the following formula (II):

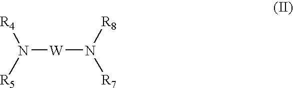

in which W is a propylene residue optionally substituted by a hydroxyl group or a C₁-C₆ alkyl radical; R₄, R₅, R₆ and R₇, which are identical or different, represent a hydrogen atom, a C₁-C₆ alkyl or C₁-C₆ hydroxyalkyl radical.

The dyeing composition in accordance with the invention may also contain, in addition to the compound(s) of formula (I) defined above, at least one additional oxidation base which may be chosen from the oxidation bases conventionally used in oxidation dyeing and among which there may be mentioned in particular para-phenylenediamines different from the compounds of formula (I), bisphenylalkylenediamines, para-aminophenols, ortho-aminophenols and heterocyclic bases.

Among the para-phenylenediamines, there may be mentioned more particularly, by way of example, para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-p-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, and their addition salts with an acid.

Among the para-phenylenediamines cited above, there are most particularly preferred para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and their addition salts with an acid.

Among the bisphenylalkylenediamines, there may be mentioned more particularly, by way of example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylene-diamine, 1,8-bis(2,5-diaminophenoxy)-3,5-dioxaoctane, and their addition salts with an acid.

Among the para-aminophenols, there may be mentioned more particularly, by way of example, para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and their addition salts with an acid.

Among the ortho-aminophenols, there may be mentioned more particularly, by way of example, 2-aminophenol, 2-amino-5-methylphenol, 2-amino-6-methylphenol, 5-acetamido-2-aminophenol and their addition salts with an acid.

Among the heterocyclic bases, there may be mentioned more particularly, by way of example, the pyridine derivatives, the pyrimidine derivatives and the pyrazole derivatives.

Among the pyridine derivatives, there may be mentioned more particularly the compounds described for example in Patents GB 1,026,978 and GB 1,153,196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their addition salts with an acid.

Among the pyrimidine derivatives, there may be mentioned more particularly the compounds described for example in German Patent DE 2,359,399 or in Japanese Patents JP 88-169,571 and JP 05 163 124, in European Patent EP 0 770 375 or Patent Application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and the pyrazolopyrimidine derivatives such as those mentioned in Patent Application FR-A-2,750,048 and among which there may be mentioned pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)-(2-hydroxyethyl)-amino]ethanol, 2-[(7-aminopyrazolo[1,5-a]pyrimidin-3-yl)-(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine, 3-amino-5-methyl-7-imidazolylpropylaminopyrazolo[1,5-a]pyrimidine, their tautomeric forms, when a tautomeric equilibrium exists, and their addition salts with an acid.

Among the pyrazole derivatives, there may be mentioned more particularly the compounds described in U.S. Pat. No. DE 3,843,892, U.S. Pat. No. DE 4,133,957 and Patent Applications WO 94/08969, WO 94/08970, FR-A-2,733,749 and DE-195 43 988 such as 4,5-diamino-1-methylpyrazole, 4,5-diamino-1-(β-hydroxyethyl)pyrazole, 3,4-diaminopyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and their addition salts with an acid.

When they are used, these oxidation bases preferably represent from 0.0005 to 12% by weight approximately of the total weight of the dyeing composition, and still more preferably from 0.005 to 6% by weight approximately of this weight.

The oxidation dyeing compositions in accordance with the invention may also contain one or more couplers and/or one or more direct dyes, in particular for modifying the shades or enriching them with glints.

The couplers which can be used in the oxidation dyeing compositions in accordance with the invention may be chosen from the couplers conventionally used in oxidation dyeing and among which there may be mentioned in particular meta-phenylenediamines, meta-aminophenols, meta-diphenols, naphthols and heterocyclic couplers such as for example indole derivatives, indoline derivatives, pyridine derivatives, indazole derivatives, pyrazolo[1,5-b]-1,2,4-triazole derivatives, pyrazolo[3,2-c]-1,2,4-triazole derivatives, benzimidazole derivatives, benzothiazole derivatives, benzoxazole derivatives, 1,3-benzodioxole derivatives and pyrazolones, and their addition salts with an acid.

These couplers are more particularly chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)-benzene, 2-amino-4-(β-hydroxyethylamino)-1-methoxybenzene, 1,3-diaminobenzene, 1,3-bis(2,4-diaminophenoxy)propane, sesamol, α-naphthol, 2-methyl-1-naphthol, 6-hydroxyindole, 4-hydroxyindole, 4-hydroxy-N-methylindole, 6-hydroxyindoline, 6-hydroxybenzomorpholine, 3,5-diamino-2,6-dimethoxypyridine, 1N-β-hydroxyethylamine-3,4-methylenedioxybenzene, 2,6-bis(β-hydroxyethylamino)toluene, 2,6-dihydroxy-4-methylpyridine, 1H-3-methylpyrazol-5-one, 1-phenyl-3-methylpyrazol-5-one, and their addition salts with an acid.

When they are present, the coupler(s) preferably represent from 0.0001 to 10% by weight approximately of the total weight of the dyeing composition and still more preferably from 0.005 to 5% by weight approximately of this weight.

The dyeing composition in accordance with the invention may also contain various adjuvants which are conventionally used in hair-dyeing compositions, such as anionic, cationic, nonionic, amphoteric or zwitterionic surfactants or mixtures thereof, anionic, cationic, nonionic, amphoteric or zwitterionic polymers or mixtures thereof, inorganic or organic thickening agents, antioxidants, penetrating agents, sequestering agents, perfumes, buffers, dispersing agents, conditioning agents such as for example silicones, film-forming agents, preservatives and opacifying agents.

Of course, persons skilled in the art will be careful to choose this or these possible additional compounds such that the advantageous properties intrinsically attached to the oxidation dyeing composition in accordance with the invention are not, or not substantially, impaired by the addition(s) envisaged.

The dyeing composition according to the invention may be provided in various forms, such as in the form of liquids, creams, gels or in any other form appropriate for carrying out a dyeing of keratinous fibers, and in particular human hair.

Another subject of the invention is the use of the compositions of the invention for the oxidation dyeing of keratinous fibers, and in particular human fibers such as hair.

The subject of the invention is also a method of dyeing keratinous fibers, and in particular human keratinous fibers such as hair, using the dyeing composition as defined above.

According to this method, at least one dyeing composition as defined above is applied to the fibers, the color being developed at acidic, neutral or alkaline pH with the aid of an oxidizing agent which is added to the dyeing composition just at the time of use or which is applied separately, simultaneously or sequentially.

According to a preferred embodiment of the dyeing method of the invention, the dyeing composition described above is preferably mixed, at the time of use, with an oxidizing composition containing, in a medium appropriate for dyeing, at least one oxidizing agent present in a sufficient quantity to develop a color. The mixture obtained is then applied to the keratinous fibers and allowed to act for 3 to 50 minutes approximately, preferably 5 to 30 minutes approximately, after which they are washed with shampoo, rinsed again and dried.

The oxidizing agent may be chosen from oxidizing agents conventionally used for the oxidation dyeing of keratinous fibers, and among which there may be mentioned hydrogen peroxide, urea peroxide, alkali metal bromates, persalts such as perborates and persulfates and enzymes among which there may be mentioned peroxidases, oxidoreductases containing 2 electrons such as uricases and oxygenases containing 4 electrons such as laccases. Hydrogen peroxide is particularly preferred.

The pH of the oxidizing composition containing the oxidizing agent as defined above is such that after mixing with the dyeing composition, the pH of the resulting composition applied to the keratinous fibers preferably varies between 3 and 12 approximately, and still more preferably between 5 and 11. It is adjusted to the desired value by means of acidifying or alkalinizing agents normally used for dyeing keratinous fibers and as defined above.

The oxidizing composition as defined above may also contain various adjuvants conventionally used in hair-dyeing compositions and as defined above.

The composition which is finally applied to the keratinous fibers may be provided in various forms, such as in the form of liquids, creams, gels, or in any other form appropriate for dyeing keratinous fibers, and in particular human hair.

Another subject of the invention is a multi-compartment device or dyeing “kit” or any other multi-compartment packaging system in which a first compartment contains the dyeing composition as defined above and a second compartment contains the oxidizing composition as defined above. These devices may be equipped with a means which makes it possible to deliver the desired mixture onto the hair, by any means known to persons skilled in the art, such as the devices described in Patent FR-2,586,913 in the name of the Applicant.

Finally, the subject of the invention is also the colored product resulting from the oxidation of at least one compound of formula (I) as defined above in the presence of at least one oxidizing agent, and optionally in the presence of at least one coupler and/or of at least one additional oxidation base.

These colored products may also be provided in the form of pigments and may be used as direct dyes for the direct dyeing of hair or may be incorporated into cosmetic products such as for example into make-up products.

The examples which follow are intended to illustrate the invention without, however, limiting the scope thereof.

### EXAMPLES

### Example 1

### [1-(4-Aminophenyl)-5-hydroxymethyl-pyrrolidin-2-yl]methanol (6)

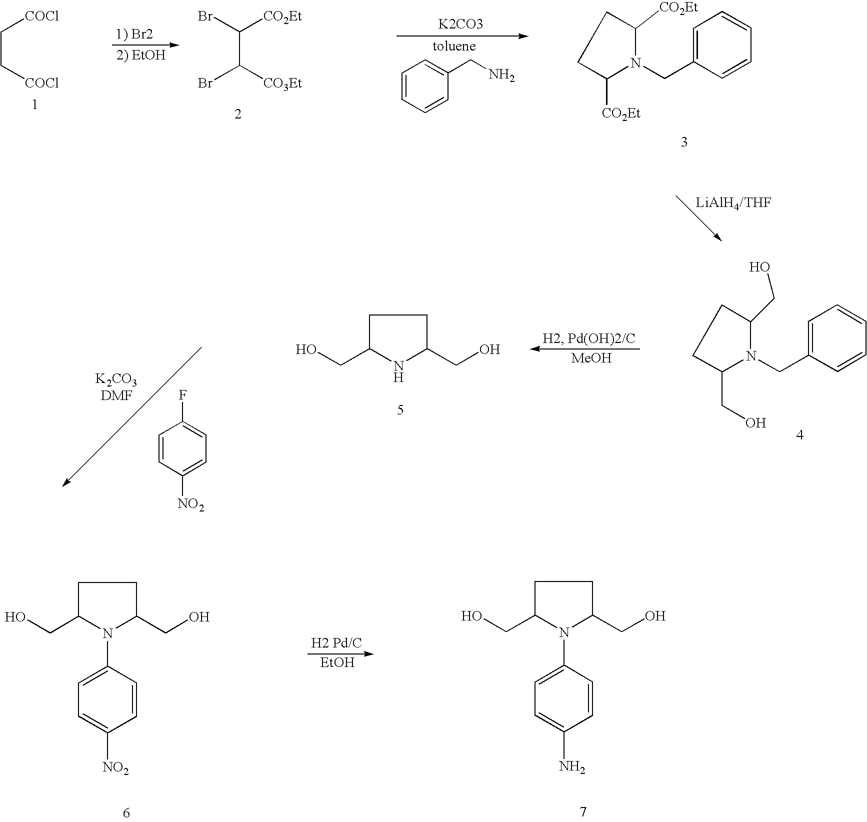

### Synthesis of Compound (2)

70 ml (1.36 mol) of bromine are added dropwise to a solution of 125 g (0.68 mol) of adipoyl chloride in 600 ml of chloroform. The solution obtained is heated for 14 h under reflux. After returning to room temperature, the mixture obtained is added dropwise to 500 ml of ethanol cooled on an ice bath, while being careful that the temperature does not exceed 50° C. After addition, the mixture is stirred overnight at room temperature and then poured into 2 l of cold water. The organic phase is collected, washed with 500 ml of a 2% sodium bisulfite solution and then twice with a 3% sodium carbonate solution. After drying over magnesium sulfate and filtration, the organic phase is concentrated under vacuum. 164 g (67%) of compound (2) are thus obtained, which compound is in the form of white crystals.

### Synthesis of Compound (3)

A mixture consisting of 120 g (0.333 mol) of compound (2), 36.4 ml (0.333 mol) of benzylamine, 115 g (0.872 mol) of potassium carbonate, 300 ml of toluene and 100 ml of water is heated for 12 hours under reflux. After decantation, the aqueous phase is extracted with 2×200 ml of ethyl ether. The organic phases are combined, dried (magnesium sulfate), filtered and then concentrated under vacuum. 57 g (56%) of compound (3) are thus obtained in the form of a yellow oil.

### Synthesis of Compound (4)

21 g (55.3 mol) of LiAlH4 are added in small fractions to a solution of 50 g (0.164 mol) of compound (3) in 400 ml of anhydrous THF, which solution is cooled on an ice bath. The mixture obtained is heated under reflux for 3 hours, cooled and then slowly poured over ice. After filtration, the solvents are removed under vacuum. 12.8 g (84%) of compound (4) are thus obtained in the form of a colorless oil.

### Synthesis of Compound (5)

25 g (0.113 mol) of compound 4, 8.6 g of palladium dihydroxide and 250 ml of methanol are placed in a reactor. Hydrogen is introduced, at atmospheric pressure, and the medium is stirred for 24 hours at room temperature. The mixture obtained is filtered and the filtrate concentrated under vacuum. 12 g (80%) of compound (5) are thus obtained in the form of a colorless oil.

### Synthesis of Compound (6)

A mixture consisting of 10 g (mol) of compound 5, 16 g (0.114 mol) of p-nitrofluorobenzene, 15.6 g (0.111 mol) of potassium carbonate and 150 ml of DMF is heated for 16 hours at 65° C. The DMF is then evaporated under vacuum and the residue is taken up in 120 ml of water. After extraction with ethyl acetate (2×200 ml), drying of the organic phase over magnesium sulfate, filtration and concentration under vacuum, a yellow solid is obtained. This product is chromatographed on silica gel, taking as eluent a chloroform/ethyl acetate 4/1 mixture. 12 g (62%) of compound 6 are thus obtained in the form of yellow crystals.

### Synthesis of Compound (7): [1-(4-aminophenyl)-5-hydroxymethylpyrrolidin-2-yl]methanol

12 g (47.6 mol) of nitro derivative 6, 400 ml of absolute ethanol and 4 g of 5% Pd/C are placed in an autoclave. A hydrogen pressure of the order of 10 bar is introduced into the apparatus and the mixture is stirred at room temperature for 7 hours. After purging with nitrogen, the catalyst is filtered and the filtrate is concentrated under vacuum. 6.5 g (73%) of compound (7) are thus obtained in the form of brown crystals.

¹H NMR (DMSO d6, 500 MHz) 1.88-1.97 (m, 4H), 3.44-3.63 (m, 6H), 6.68 (m, 4H)

Mass spectrum: spectrum in agreement with the structure

### Example 2

### Dyeing Compositions

The following dyeing compositions in accordance with the invention were prepared:

| Examples | 1 | 2 | 3 | 4 |
|---|---|---|---|---|
| 1-(4-Aminophenyl)-5-hydroxy- | 6 10⁻³ | 6 10⁻³ | 6 10⁻³ | 6 10⁻³ |
| methylpyrrolidin-2-yl]methanol | mol | mol | mol | mol |
| Resorcinol | | 6 10⁻³ | | |
| | | mol | | |
| 1-Methyl-4-aminophenol | | | 6 10⁻³ | |
| | | | mol | |
| 1-β-Hydroxyethyloxy-2,4-diamino- | | | | 6 10⁻³ |
| benzene, 2HCl | | | | mol |
| Common dye carrier | (*) | (*) | (*) | (*) |
| Water qs | 100 g | 100 g | 100 g | 100 g |

| | | |
|---|---|---|
| (*) Common dye carrier: | | |
| Polyglycerolated oleyl alchohol | 4.0 | g |
| containing 2 mol of glycerol | | |
| Polyglycerolated oleyl alchohol | 5.69 | g A.S. |
| containing 4 mol of glycerol, containing | | |
| 78% of active substances (A.S.) | | |
| Oleic acid | 3.0 | g |
| Oleyl amine containing 2 mol of ethylene | 7.0 | g |
| oxide, sold under the trade name | | |
| ETHOMEEN O12 ® by the company AKZO | | |
| Diethylaminopropyl laurylamino | 3.0 | g A.S. |
| succinamate, sodium salt containing 55% | | |
| of A.S. | | |
| Oleyl alcohol | 5.0 | g |
| Oleic acid diethanolamide | 12.0 | g |
| Propylene glycol | 3.5 | g |
| Ethyl alcohol | 7.0 | g |
| Dipropylene glycol | 0.5 | g |
| Propylene glycol monomethyl ether | 9.0 | g |
| Sodium metabisulfite as an aqueous | 0.455 | g A.S. |
| solution containing 35% of A.S. | | |
| Ammonium acetate | 0.8 | g |
| Antioxidant, sequestrant | q.s. | |
| Perfume, preservative | q.s. | |
| Aqueous ammonia containing 20% of NH₃ | 10.0 | g |

Polyglycerolated oleyl alcohol 4.0 g containing 2 mol of glycerol

Polyglycerolated oleyl alcohol 5.69 g A.S. containing 4 mol of glycerol, containing 78% of active substances (A.S.)

Oleic acid 3.0 g

Oleyl amine containing 2 mol of ethylene 7.0 g oxide, sold under the trade name ETHOMEEN O12® by the company AKZO

Diethylaminopropyl laurylamino 3.0 g A.S. succinamate, sodium salt containing 55% of A.S.

Oleyl alcohol 5.0 g

Oleic acid diethanolamide 12.0 g

Propylene glycol 3.5 g

Ethyl alcohol 7.0 g

Dipropylene glycol 0.5 g

Propylene glycol monomethyl ether 9.0 g

Sodium metabisulfite as an aqueous 0.455 g A.S. solution containing 35% of A.S.

Ammonium acetate 0.8 g

Antioxidant, sequestrant q.s.

Perfume, preservative q.s.

Aqueous ammonia containing 20% of NH₃ 10.0 g

At the time of use, each dyeing composition is mixed with an equal quantity of an oxidizing composition consisting of a solution of hydrogen peroxide at 20 volumes (6% by weight) and having a pH of about 3.

Each mixture obtained has a pH of about 9.5 and is applied for 30 minutes to locks of natural gray hair which is 90% white.

After rinsing, washing with shampoo, rinsing and drying, each lock is evaluated before and after dyeing in the L*a*b* system, by means of a spectrophotometer CM 2002 MINOLTA®, (Illuminant D65).

In the L*a*b* system, the three parameters denote the intensity (L*), the shade (a*) and the saturation (b*), respectively. According to this system, the higher the value of L, the lighter or less intense the color. Conversely, the lower the value of L, the darker or more intense the color. a* and b* indicate two color axes, a* indicates the green/red color axis and b* the blue/yellow color axis. The results are presented in the following table

| | L* | a* | b* |
|---|---|---|---|
| Ex. 1 | 48.20 | 2.63 | 6.79 |
| Ex. 2 | 33.5 | 0.4 | −4.9 |
| Ex. 3 | 29.5 | −0.5 | −13.3 |
| Ex. 4 | 26.2 | −3.34 | −11.47 |

1. A composition for the oxidation dyeing of keratinous fibers, and in particular human keratinous fibers such as hair, comprising, in a medium appropriate for dyeing,
at least one oxidation base chosen from compounds of the following formula (I), and/or their addition salts with an acid 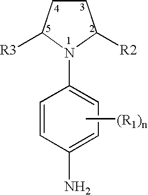
in which:
R₁ represents a halogen atom; a linear or branched C₁-C₇ carbon chain, which is saturated or which may contain one or more double bonds and/or one or more triple bonds, which may be in the form of a 3- to 6-membered ring, it being possible for one or more carbon atoms of the chain to be replaced by an oxygen, nitrogen or sulfur atom, by an SO₂ group or by a halogen atom, the radical R₁ not containing a peroxide bond, or a diazo, nitro or nitroso radical;
R₂ and R₃ represent, independently of each other, a saturated or unsaturated C₁-C₄ carbon chain; a C₁-C₄ alkyl radical substituted with a C₁-C₄ alkoxy radical, an acetoxy radical, an amino radical, a carboxyl radical, a carbamoyl radical, a (C₁-C₄) mono- or dialkylcarbamoyl radical, a (C₁-C₄)alkoxycarbonyl radical, a C₁-C₆ monohydroxyalkoxy radical or with a C₂-C₆ polyhydroxyalkoxy group; a C₁-C₄ monohydroxyalkyl radical; a C₁-C₆ polyhydroxyalkyl radical; a C₁-C₄ aminoalkyl radical in which the amine is mono- or disubstituted with a C₁-C₄ alkyl radical, an acetyl radical, a C₁-C₄ monohydroxyalkyl radical or a C₂-C₆ polyhydroxyalkyl radical; a C₂-C₄ polyaminoalkyl radical; a C₂-C₄ alkyl radical substituted with at least one amino group and at least one hydroxyl group; a carboxyl radical; a carbamoyl radical; a (C₁-C₄) mono- or dialkylcarbamoyl radical; a (C₁-C₄)alkoxycarbonyl radical; a (C₁-C₄)alkylcarbonyl radical
n is between 0 and 2, it being understood that when n is equal to 2, then the radicals R₁ may be identical or different, with the exception of the compound 1-(3-isopropyloxy-4-aminophenyl)-2,5-dimethylpyrrolidine and the compound 1-(3-methyl-4-aminophenyl)-2,5-dihydroxymethylpyrrolidine.

2. The composition as claimed in claim 1, in which the radical R₁ of formula (I) is chosen from a chlorine or bromine atom, a methyl, ethyl, isopropyl, vinyl, allyl, methoxymethyl, hydroxymethyl, 1-carboxymethyl, 1-aminomethyl, 2-carboxyethyl, 2-hydroxyethyl, 3-hydroxypropyl, 1,2-dihydroxyethyl, 1-hydroxy-2-aminoethyl, 1-amino-2-hydroxyethyl, 1,2-diaminoethyl, methoxy, ethoxy, allyloxy, or 2-hydroxyethyloxy radical.

3. The composition as claimed in claim 1 or 2, in which the radicals R₂ and R₃ are chosen from the hydroxymethyl radical, aminomethyl radical, carboxyl radical, carbamoyl radical, 2-hydroxyethyloxymethyl radical, 2-hydroxyethylaminomethyl radical, and methoxymethyl.

4. The composition as claimed in any one of claims 1 to 3, in which n is 0 or 1.

5. The composition as claimed in any one of claims 1 to 4, in which the compound of formula (I) is chosen from the group consisting of:

| Formula | Nomeclature | Formula | Nomeclature |
|---|---|---|---|
| 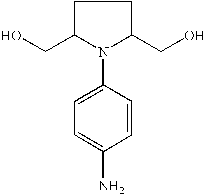 | [1-(4-Aminophenyl)-5- hydroxymethylpyrrolidin-2- yl]methanol | 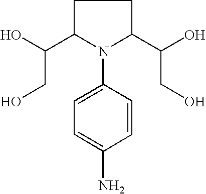 | 1-[1-(4-Aminophenyl)-5- (1,2-dihydroxy- ethyl)pyrrolidin-2-yl]- ethane-1,2-diol |
| 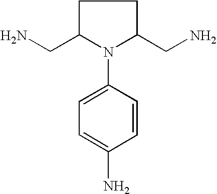 | 4-(2,5-Bisamino- methylpyrrolidin-1-yl)- phenylamine | 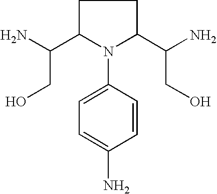 | 2-Amino-2-[5-(1-amino-2- hydroxyethyl)-1-(4- aminophenyl)pyrrolidin-2- yl]ethanol |
| 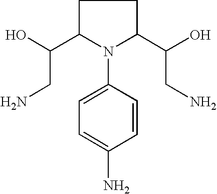 | 2-Amino-1-[5-(2-amino-1- hydroxyethyl)-1-(4- aminophenyl)pyrrolidin-2- yl]ethanol | 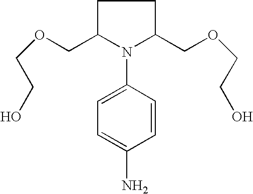 | 2-[1-(4-Aminophenyl)-5-(2- hydroxyethoxy- methyl)pyrrolidin-2- ylmethoxy]ethanol |
| 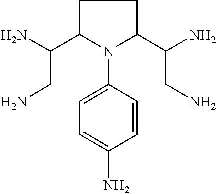 | 1-[1-(4-Aminophenyl)-5- (1,2-diaminoethyl)- pyrrolidin-2-yl]ethane-1,2- diamine | 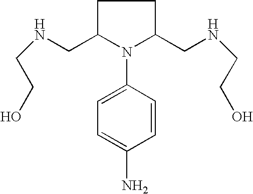 | 2-({1-(4-Aminophenyl)-5- [(2-hydroxyethyl- amino)methyl]pyrrolidin-2- ylmethyl}amino)ethanol |
| 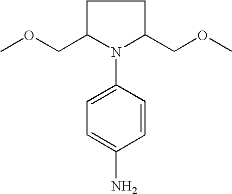 | 4-(2,5-Bismethoxy- methylpyrrolidin-1- yl)phenylamine | 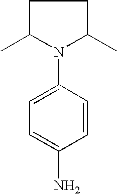 | 4-(2,5-Dimethylpyrrolidin- 1-yl)phenylamine |
| 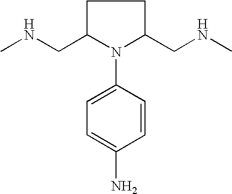 | 4-(2,5-Bismethyl- aminomethylpyrrolidin-1- yl)phenylamine | 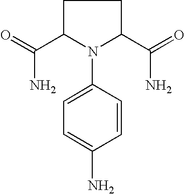 | 1-(4-Aminophenyl)- pyrrolidin-2,5-dicarboxylic acid diamide |
| 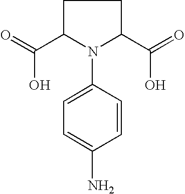 | 1-(4-Aminophenyl)- pyrrolidin-2,5-dicarboxylic acid | 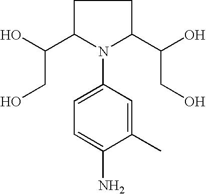 | 1-[1-(4-Amino-3-methyl- phenyl)-5-(1,2- dihydroxyethyl)pyrrolidin- 2-yl]ethane-1,2-diol |
| 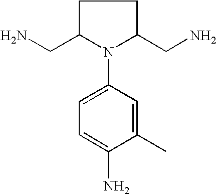 | 4-(2,5-Biamino- methylpyrrolidin-1-yl)-2- methylphenylamine | 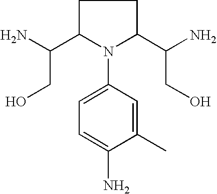 | 2-Amino-2-[5-(1-amino-2- hydroxyethyl)-1-(4-amino- 3-methylphenyl)ptrrolidin- 2-yl)ethanol |
| 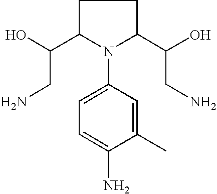 | 2-Amino-1-[5-(2-amino-1- hydroxyethyl)-1-(4-amino- 3-methylphenyl)pyrrolidin- 2-yl)ethanol | 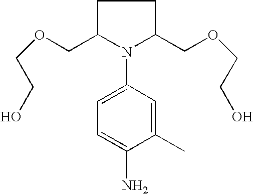 | 2-[1-(4-Amino-3-methyl- phenyl)-5-(2-hydroxy- ethoxymethyl)pyrrolidin-2- ylmethoxy)ethanol |
| 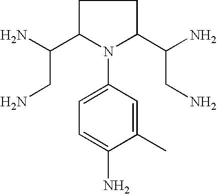 | 1-[1-(4-Amino-3- methylphenyl)-5-(1,2- diaminoethyl)pyrrolidin-2- yl]ethane-1,2-diamine | 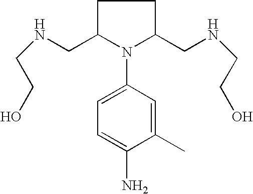 | 2-({1-(4-Amino-3-methyl- phenyl)-5-[(2-hydroxyethyl- amino)methyl]pyrrolidin-2- ylmethyl)amino)ethanol |
| 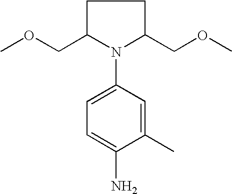 | 4-(2,5-Bismethoxymethyl- pyrrolidin-1-yl)-2-methyl- phenylamine | 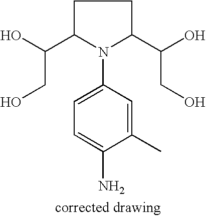 | 1-[1-(4-Amino-3-methyl- phenyl)-5-(1,2-dihydroxy- ethyl)pyrrolidin-2- yl]ethane-1,2-diol |
| 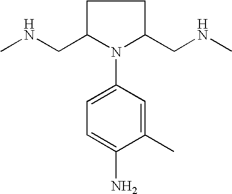 | 4-(2,5-Bismethylamino- methylpyrrolidin-1-yl)-2- methylphenylamide | 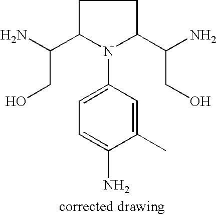 | 2-Amino-2-[5-(1-amino-2- hydroxyethyl)-1-(4-amino- 3-methylphenyl)pyrrolidin-0 2-yl]ethanol |
| 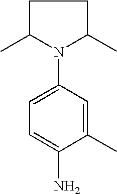 | 4-(2,5-Dimethylpyrrolidin- 1-yl)-2-methylphenylamine | 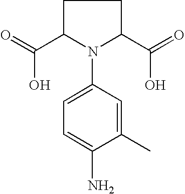 | 1-(4-Amino-3- methylphenyl)pyrrolidine- 2,5-dicarboxylic acid |
| 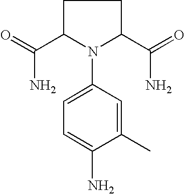 | 1-(4-Amino-3-methyl- phenyl)pyrrolidin-2,5- dicarboxylic acid diamide | | |

and their addition salts with an acid.

6. The composition as claimed in any one of claims 1 to 5, characterized in that the compound(s) of formula (I) represent from 0.0005 to 12% by weight of the total weight of the dyeing composition.

7. The composition as claimed in any one of claims 1 to 6, characterized in that it contains, in addition to the compound(s) of formula (I), at least one additional oxidation base chosen from para-phenylenediamines different from the compounds of formula (I), bis-phenylalkylenediamines, para-aminophenols, ortho-aminophenols and heterocyclic bases.

8. The composition as claimed in any one of claims 1 to 7, characterized in that the additional oxidation base(s) represent from 0.0005 to 12% by weight of the total weight of the dyeing composition.

9. The composition as claimed in any one of claims 1 to 8, characterized in that it contains at least one coupler and/or at least one direct dye.

10. The composition as claimed in claim 9, characterized in that the couplers are chosen from meta-phenylenediamines, meta-aminophenols, meta-diphenols, naphthols and heterocyclic couplers.

11. The composition as claimed in either of claims 9 and 10, characterized in that the coupler(s) represent from 0.0001 to 10% by weight of the total weight of the dyeing composition.

12. The composition as claimed in any one of claims 1 to 11, characterized in that the addition salts with an acid of the compounds of formula (I) are chosen from the hydrochlorides, hydrobromides, sulfates, citrates, succinates, tartrates, lactates and acetates.

13. The use of the composition as claimed in any one of claims 1 to 12 for the oxidation dyeing of keratinous fibers, and in particular human fibers such as hair.

14. A method for the oxidation dyeing of keratinous fibers, and in particular human keratinous fibers such as hair, characterized in that the composition as defined in any one of claims 1 to 12, and an oxidizing agent are applied to the fibers, the oxidizing agent being added to the composition at the time of use or applied to the fibers separately, simultaneously or sequentially.

15. The method as claimed in claim 14, characterized in that the oxidizing agent is chosen from hydrogen peroxide, urea peroxide, alkali metal bromates, persalts, peracids, and enzymes.

16. A multicompartment device comprising a first compartment containing the composition as defined in any one of claims 1 to 12 and a second compartment containing an oxidizing composition.

17. A colored product which can be obtained by reacting the composition as defined in any one of claims 1 to 12 with an oxidizing agent.
